(19) United States
(12) Patent Application Publication
(10) Pub. No.: US 2021/0096132 A1
(43) Pub. Date: Apr. 1, 2021

(54) System and Method for Nano Cancer Cell Sorting Printer

(71) Applicant: Nano Medic Norge AS, Verdal (NO)

(72) Inventor: Mehrdad SALEHMANESH, Trondheim (NO)

(21) Appl. No.: 16/955,408

(22) PCT Filed: Dec. 20, 2018
(86) PCT No.: PCT/NO2018/050321

§ 371 Date: Jun. 18, 2020

(30) Foreign Application Priority Data
Dec. 20, 2017 (NO) 20172019
Mar. 27, 2018 (DE) 10 2018 107 258.9

(51) Int. Cl. G01N 33/574; G01N 1/28; B01L 3/00; G01N 15/06

(52) U.S. Cl. CPC G01N 33/57492; G01N 1/2813; B01L 3/502761; G01N 2015/0693; G01N 2446/00; B01L 2200/0652; B01L 2400/043; G01N 15/0612

(57) ABSTRACT

A system and method for sorting and printing of cancer cells in general and more specifically a system and a method for enriching cancer cells in a sample using magneto-phoresis and cytology rotor in cell centrifuge using cytospin method. The present invention comprises a technological advantage over known systems and methods by use of a dynamic system wherein a fluid flow comprising cells flow across a magnetic field to separate cells based on absence or presence of paramagnetic nano particles attached to them.

### BACKGROUND OF THE INVENTION

### Field of the Invention

The invention relates to a system and method for enrichment of cells based on their surface antigen characteristics in general and more specifically a system and a method for enriching cancer cells in a sample using a combination of magneto-phoresis and cytology rotor and cyto clips.

### Background Art

State of the art is reflected in column cell sorting techniques. However so far these techniques could not be put in the practice in pathology laboratories clinics due to below limitations:
limitations in specimen volumes (not efficient in microscales)
limited sample throughput and processing speeds that would make processing clinical scale samples unfeasible
high operating pressures that could result in a loss of function or viability of cells
bulky instrumentation that occupy large bench footprints, technical expertise necessary for operating complex machinery
increased risk of sample contamination and safety concerns due to the sorting of aerosolized samples
There is no method that directly sends the target cells on the glass slide for pathologic studies and microscopic evaluation.

Also Dynal magnetic microbeads, Miltenyi magnetic nanobeads, and fluorescence-activated cell sorting cytometry (FACS) are well known for cell sorting. However, the current immunomagnetic methods are based on static attraction of magnetically labelled cells in a tube placed on a magnet (Dynal), or by passing cells through a magnetic column matrix (Miltenyi). Neither method has changed in 30 years and has severe limitations on throughput and performance, specifically in very small volumes in microliter scale. Furthermore the demand for cell sorting is quickly growing to the isolation of target cell populations, such as circulating tumour cells (CTCs), hematopoietic stem cells (HSCs), and circulating foetal cells (CFCs) from blood. This kind of target cell isolation is demanding a continuous flow method that performs well in microscales which has been provided by this invention. In the pathology laboratories there is only one active indirect competitor for women uterus cervical smear test (namely Pap smear) in the name of Thinprep which is just providing better quality for viewing and has nothing to do with cell sorting based on the biomarkers.

From prior art one should refer to U.S. Pat. No. 9,535,036 relating to a multiple discrimination device having a three-dimensional micro ferromagnetic pattern to allow a magnetic force applied to a discrimination-target particle.

Reference is also made to US 2016016180 A1 relating to a device and a method that uses acoustic waves and magnetic forces to separate magnetically labelled particles.

The scientific publication «Enrichment of rare cancer cells through depletion of normal cells using density and flow-through, immunomagnetic cell separation” by Lara O. et al describes enrichment and detection of rare cancer cells in blood suspension using negative selection steps including a flow-through immunomagnetic cell separation system and by optimizing variables normally encountered during such enrichment processes. After the enrichment, the cells are analysed either at automated cell count, filtration and visual counting, or by cytospin analysis.

US 2003170609 A1 describes a method for selecting particles having a predetermined property from a population of a multiplicity of different particles and to a device suitable for carrying out said method.

US 2012135494 A1 relates to a magnetic separator comprising a separation chamber. The magnetic separator comprises an inlet and at least one outlet, and a magnetic source operatively coupled to the separation chamber and comprising a plurality of magnets that can be selectively turned off and on to create a dynamic magnetic field in the separation chamber.

The publication US 2012115755 A1 describes a microfluidic device that may employ one or more sorting stations for separating target species from other species in a sample. The separation is driven by magnetophoresis. A sorting station generally includes separate buffer and sample streams. A magnetic field gradient applied to the sorting station deflects the flow path of magnetic particles (which selectively label the target species) from a sample stream into a buffer stream. The buffer stream leaving the sorting station is used to detect or further process purified target species labeled with the magnetic particles.

These publications are not practical in clinical cytology laboratories due to low sensitivity, a high number of false negative results and a low throughput. They do not provide for cytology labs' supervisors (Pathologists) any visual control on the test results which causes conflicts over legal approval of the diagnosis. There is therefore a need for a method and a system to overcome these problems while providing pathologists a device to help for faster decision making for biopsy tests with better sensitivity and less false negative results in hospitals and clinical laboratories.

### SUMMARY OF THE INVENTION

### Problems to be Solved by the Invention

Therefore, a main objective of the present invention is to provide a system and method for enrichment of cancer cells from clinics' biopsy samples and print them on the glass slide for microscopic evaluation which provides more sensitivity and less false negative test results for cancer studies in pathology laboratory.

### Means for Solving the Problems

The objective is achieved according to the invention by an apparatus for enrichment of cancer cells as defined in the preamble of claim 1, having the features of the characterising portion of claim 1 and.

A number of non-exhaustive embodiments, variants or alternatives of the invention are defined by the dependent claims.

The present invention attains the above-described objective by using a Y-shaped microfluidic separator in a magnetic field and printing the cells from the test and control tubes on the test and control microscopic glass slide.

### Effects of the Invention

The present invention comprises a technological advantage over known systems and methods by use of a dynamic system wherein a fluid flow comprising tagged cells (tagged with conjugated nano beads) flow across a magnetic field to separate cells based on absence or presence of related antigens on their surface which matches with paramagnetic particles' antibody and then print the cells on the surface of glass microscopic slides for further pathological analysis.

These effects provide in turn several further advantageous effects:
faster sorting rates
an ability to process native biological fluids
an ability to process diverse cell types
enhanced capabilities for multiplexed sorting
simpler operating procedures enabling fully automated systems
reduced biohazard risk by eliminating aerosols
reduced cost
reduced size for operational convenience and portability
providing high sensitivity and less false negative for cancer cytology tests in clinical laboratory
providing opportunity for making a high throughput device which could print more than five samples at one run
By adding a commercial lens free holographic imaging compartment (such as CMOS) to the end of process and with algorithmic analysis of image, the ratio between the target cells in test slide to normal cells in control slides can be calculated, which gives an understanding about the stage of tumor malignancy and plan for personal treatment.

### BRIEF DESCRIPTION OF THE DRAWINGS

The above and further features of the invention are set forth with particularity in the appended claims and together with advantages thereof will become clearer from consideration of the following detailed description of an [exemplary] embodiment of the invention given with reference to the accompanying drawings.

The invention will be further described below in connection with exemplary embodiments which are schematically shown in the drawings, wherein:

FIG. 1 shows an embodiment of a Y-shaped separator,

FIG. 2 shows an embodiment of a compound separator system,

FIG. 3 shows an embodiment of a system comprising a Y-shaped separator,

FIG. 4 shows a cartridge, and

FIG. 5 shows a lens free cell counter analyzer CMOS

### DESCRIPTION OF THE REFERENCE SIGNS

The following reference numbers and signs refer to the drawings:

| | |
|---|---|
| 10 | The system |
| 100 | The device for enrichment of cells |
| 102 | Input fluid |
| 104 | Fluid subjected to a magnetic field |
| 106 | Pump |
| 110 | A first fluid stream |
| 120 | A second fluid stream |
| 140 | Magnetic field |
| 142 | Magnet |
| 200 | A separator |
| 202 | Separator input |
| 204 | Separator tube |
| 210 | A first output tube |
| 220 | A second output tube |
| 300, 400 | Second and third separators |
| 310, 410 | A first output stream from the second and third separators |
| 320, 420 | A second output stream from the second and third separators |
| 510 | First sample tube, control tube |
| 520 | Second sample tube, test tube |
| 600 | Centrifuge, cytospin |
| 710 | First slide, control slide |
| 720 | Second slide, test slide |
| 800 | CMOS lens free imaging compartment |
| 810 | Slides |
| 820 | Led |
| 830 | Pinhole |

### DETAILED DESCRIPTION OF THE INVENTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

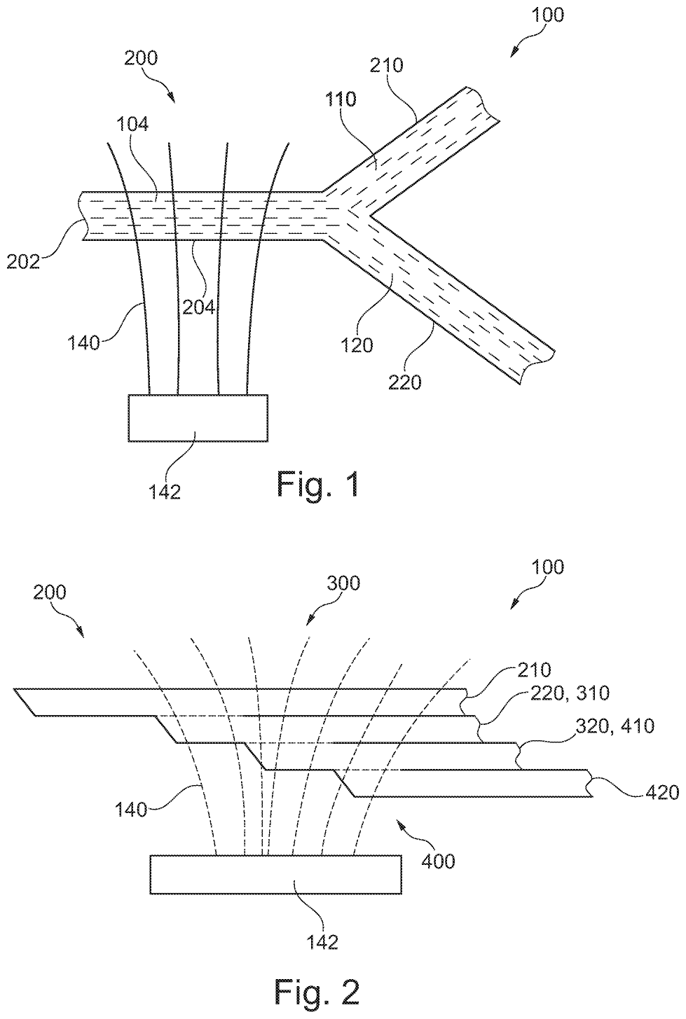

The invention will be further described in connection with exemplary embodiments, which are schematically shown in the drawings, wherein FIG. 1 shows an embodiment of a Y-shaped separator which is part of the overall separator system.

In terms of terminology, it should be noted that tagged and targeted calls are essentially the same. In starting the process, the term target cells are often used and in the machine these become tagged. This is a matter of name and conventions, of material difference.

Principles Forming the Basis of the Invention

The underlying principle of the invention is that a magnetic field can be used to discriminate between tagged cancer cells with paramagnetic particles in a continuous cytometric system to separate one stream into a first enriched stream and a second depleted stream. This results in continuous-flow separation, to draw or deter particles or target cells from their normal path in a fluid flow, and send them to a particular outlet for pathology tests and analysis.

FIG. 1 shows part of an embodiment of a system 100 having a Y-shaped separator 200 having an input 202, a tube 204 for fluid 104, a junction 206 from which a first tube 210 for a first fluid stream 110 and a second tube 220 for a second fluid stream 120 extend. A magnetic field 140 acts upon the fluid 104 in the tube 204 thus separating the fluid stream into a first and a second fluid stream. Typically the fluid stream closest to the magnet is enriched in terms of cells having a magnetic agent connected to antibody markers on the cells.

A nano paramagnetic beads which has been conjugated with antibodies attached to a cancer cell or any cell with pathologic characteristics antigens on their surface, enabling magneto-phoresis, or the movement of a particle by force of a magnetic field. The magnetic agent can be used to select cells for concentrating or enriching a stream, or for negative selection to remove cells from a stream. In case of negative selection, the cells which presenting a specific disease would not show normal biomarkers on their surface. As a result by using nano beads which cover with antibody for normal biomarkers we separate the normal cells and the remained are the cells which due to a disease do not show the normal surface biomarkers.

A second underlying principle is that such separators can be connected serially to further enrich a stream.

FIG. 2 shows a compound system having a first 200, second 300 and third 400 separators connected in series. A magnet subjects the separators to a magnetic field 140. In this example a positive selection is employed where it is desired to concentrate a fluid stream of pathologic cells having a paramagnetic nano particles connected to antibodies.

An enriched output from the first separator 200 continues into a second separator 300, the enriched output of which continues to a third separator 400, the output of which is used for analysis.

After single or compound enrichment the output is available for entering into the test and control tubes that already attached to glass slides by cytoclips inside a cytology rotor which print the cells on microscopic glass slides for pathologist view and analysis by using G force. A depleted output is prepared as a control sample to verify the correct operations of the system.

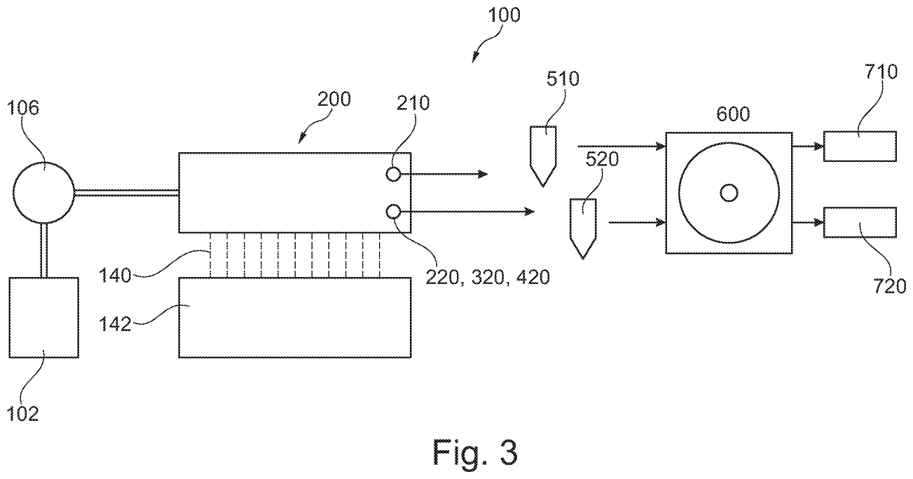

FIG. 3 shows an embodiment of a system 100 comprising a Y-shaped separator. A reservoir holds the input fluid 102 with pathologic cells having a paramagnetic nanoparticles connected to the cells' antibodies. A pump 106 pumps the fluid from the reservoir into the separator 200, thus ensuring that the fluid crosses the magnetic field 140 set up by a strong magnet 142. After passing through the separator the cells in the fluid are sorted into a first output tube 210. Being furthest from the magnet this is fluid depleted in pathologic cells having a paramagnetic nanoparticles connected to the cells' antibodies. Pathologic cells enriched by the separator are sorted into a second output tube 220 for a single separator or output tube 320, 420 for a compound separator, and then transferred to a test tube 520. The test tube is transferred to a centrifuge 600 which is preferably a using cytospin method, for further enrichment. The enriched cells are then printed to a test slide 720.
Preferably fluid from the first output tube 210, depleted in pathological cells, is transferred to a control tube 510. The control tube is transferred to the cytology rotor to centrifuge and printed to a control slide 710.

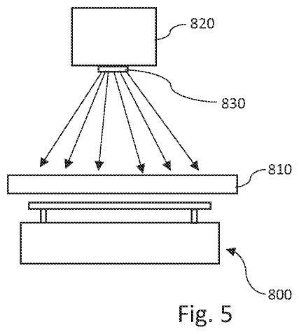

FIG. 5. After printing the microscope slides, both test and control slides carry manually to CMOS lens free compartment (800) for analyzing the cells' characteristics and giving the number of cells and the cells number ratio between the test slide and control slides.

### BEST MODES OF CARRYING OUT THE INVENTION

In a preferred embodiment the system is adapted for cancer cell sorting, operating as a screener for cytology laboratory. In practical use a sample of cells is extracted from a body, for instance from lymph nodes or breast tumour using a biopsy needle or by brushing an expose area of an organ like uterus cervix. The samples are treated with magnetic anti bodies dependent on the type of samples. So far there are below antibodies commercially available for separating abnormal cells:
Bone marrow: 35 antigens, 697 commercial products
Plasma cells: 28 antigens, 613 commercial products
Lung cancer: 5 antigens, 44 commercial products

There are also many others that shares among wide range of cancers, like anti-EpCAM & anti-Mucin.

One can also employ negative selection method. For negative selection to remove cells from a stream. In case of negative selection, the cells which presenting a specific disease would not show normal biomarkers on their surface. As a result by using nano beads which cover with antibody for normal biomarkers we separate the normal cells and the remained are the cells which due to a disease do not show the normal surface biomarkers.

The process starts with samples obtained from an organism. The volume can be as little as 100 microliter. Samples are placed in tubes and centrifuged at 500 g for 5 minutes. The sample is re-suspended per 50 μl of buffer solution (PBS+1% FCS). Next 10 μl of a nano bead solution coated with antibody is added. Typically the tube is kept on ice for 20-30 minutes, depending on the type of antibody and cells. Next the cells are washed by adding 1-2 ml of buffer and centrifuged at 300 g for 5 minutes. Finally the cells are re-suspended with 0.5 ml buffer for sorting.

The cells are prepared and entered into the separator cartridge and the enriched cancer cells are extracted from the output of the separator into a test tube brought After single or compound enrichment the output is available for entering into the test tube that already attached to glass slide by cytoclips inside a cytology rotor which print the cells on microscopic glass slides for pathologist view and analysis by using G force.

It is also preferred to also recover cells from the depleted output of the separator into a control tube and process this with cytospin method as with the test tube with enriched cells. This result in a second cytology slide for viewing by a pathologist, allowing a control to ensure that the correct procedure was followed, so that for instance an incorrect antibody or wrong technical method is not used.

The preferred separator system is a quartz (or any resistant material) microfluidic chip used as the separator for easy cleaning and multiple usage. However in case of using for clinical samples polymer disposable separators will be used that easily can be change after each usage, for sake of sterilization and cell contamination prevention from previous samples. A magnetic field 140 is set up using a strong permanent magnet. It is preferred to use a compound separator system. While a series of Y-shaped separators can be used it is envisaged that a system of branching parallel tubes are used where at each successive branch cancer cells are concentrated in the direction of the magnet by attraction as the fluid cell suspension traverses the magnetic field. With three separators in series there will be greatly improved separation

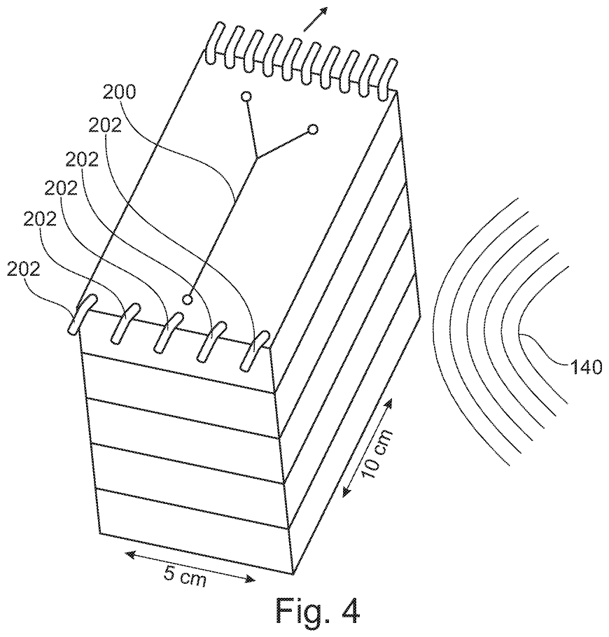

For sorting a microfluidic cartridge is preferably used having a separator in the form of a Y-shaped channel. The tubes are typically 150 μm deep where the separator tube 204 is 165 μm wide while output tubes 210, 220, 320, and 420 are 60-75 μm wide. FIG. 4 shows such microfluidic cartridges comprising a plurality, typically five, Y-shaped separators.

### Alternative Embodiments

A number of variations on the above can be envisaged. For instance the magnet can be a permanent magnet or an electromagnet.

### INDUSTRIAL APPLICABILITY

The invention according to the application finds usage in quickly and efficiently concentrating cancer or any pathologic cells printed on a microscopic glass slides for microscopic analysis by for instance a pathologist.

The method brings a high sensitivity to the cytology test results and less false negative that consequently will reduce late diagnosis and higher cost of the treatments

The method can sort and print more than five samples in each run which reduces the labour cost in clinical laboratory

The high throughput ability of the method also is very helpful for epidemiologic studies when many samples from a community are taken to clinical cytology laboratory for searching a specific disease.

The cell counting, and analysis of test and control slides provides the ratio of number of cancer cells to normal cells which opens a new era in personal treatment since it shows the malignancy grade of a tumor individually.

1. A method for enrichment of cells having paramagnetic nanoparticles connected to the cells' antibodies, in a fluid (102, 104), using a system, comprising one or more separators (200, 300, 400),
wherein the separator comprises:
an input (202),
a tube (204) for the fluid (104),
a junction (206) from which
a first tube (210) fora first fluid stream (110) and
a second tube (220, 320, 420) for a second fluid stream (120) extend,
wherein a magnetic field (140) acts upon the paramagnetic nanoparticles in the fluid (104) in the tube (204) as the fluid crosses the magnetic field, thus separating the fluid stream into the first (110) and the second (120) fluid stream, the method comprising the steps of:
a) entering a sample into the input (202),
b) separating the sample in the separator (200, 300,400) by pumping the sample along the tube (202) subjected to the magnetic field (140),
c) extracting fluid enriched in cells from an enriched fluid stream output of the separator into a test tube,
d) processing the test tube sample in a centrifuge and printing the processed test tube sample to a glass slide for analysis.

2. The method according to claim 1, wherein step d) is performed in a Cytospin.

3. The method according to claim 1, further comprising the steps
e) extracting fluid depleted in cells from a depleted fluid stream output of the separator into a control tube,
f) processing the control tube sample in a centrifuge and printing the processed control tube sample to a glass slide for analysis.

4. The method according to claim 3, wherein steps d) and f) are performed in a Cytospin.

5. The method according to any of the previous claims, further comprising the steps
i) putting the printed test slides, and processing them, in a lens free holographic imaging system, comprising a light beam (820), a pinhole (830), a slides chamber (810) and an optical sensor (800), displaying the cells characteristics, and
ii) calculate the ratio between the target cells in the test slide to the normal cells in the control slides by means of algorithmic image analysis in the optical sensor

6. The method according to claim 5 wherein the printed test slides are put manually in the lens free holographic imaging system.

7. The method according to claim 5 or 6 wherein the optical sensor is a CMOS sensor.

8. A system (10) for sorting cells having paramagnetic nanoparticles connected to the cells' antibodies, in a fluid (102, 104), comprising:
a device (100) for enrichment of cells comprising one or more separators (200, 300, 400),
wherein the separator comprises:
an input (202),
a tube (204) for the fluid (104),
a junction (206) from which
a first tube (210) for a first fluid stream (110) and
a second tube (220, 320, 420) for a second fluid stream (120) extend,
wherein a magnetic field (140) acts upon the paramagnetic nanoparticles in the fluid (104) in the tube (204) as the fluid crosses the magnetic field, thus separating the fluid stream into the first (110) and the second (120) fluid stream
a pump (106) for pumping fluid from a reservoir into the device (100) for enrichment of cells,
a magnet (142) for setting up a field (140) that the fluid crosses, and
a centrifuge (600) for enriching cells in the fluid, and means for processing the test tube sample in a centrifuge and characterized by means for printing the processed test tube sample to a glass slide.

9. A system (10) according to claim 8 wherein the system further comprises a lens free holographic imaging system, comprising a light beam (820), a pinhole (830), a slides chamber (810) and an optical sensor (800).
